(19) United States
(12) Patent Application Publication
(10) Pub. No.: US 2014/0094838 A1
(43) Pub. Date: Apr. 3, 2014

(54) DEVICE SYSTEM AND METHOD FOR BLUNT TISSUE DISSECTION

(71) Applicant: POP MEDICAL SOLUTIONS LTD., Tel-Aviv (IL)

(72) Inventors: Menahem Neuman, Karmei Yosef (IL); Eyal Sandach, Yahud (IL); Boaz Harari, Ganei-Tikva (IL); Harel Gadot, New York, NY (US)

(21) Appl. No.: 14/125,590

(22) PCT Filed: Jun. 12, 2012
(86) PCT No.: PCT/IL2012/050201

§ 371 Date: Dec. 12, 2013

Related U.S. Application Data
(60) Provisional application No. 61/496,064, filed on Jun. 13, 2011.
(60) Provisional application No. 61/548,278, filed on Oct. 18, 2011.

(51) Int. Cl. A61B 17/29

(52) U.S. Cl. CPC A61B 17/29
USPC 606/190

(57) ABSTRACT

A surgical device, and a system and method using same are provided. The surgical device includes an elongated device body attached to a dissection head having a tissue biasing mechanism configured for blunt tissue dissection and an element for preventing trapping of tissue within the tissue biasing mechanism. According to still further features in the described preferred embodiments the element for preventing trapping of tissue between the at least one pair of movable jaws is a finger-like extension positioned between the at least one pair of movable jaws.

### FIELD AND BACKGROUND OF THE INVENTION

The present invention relates to a device, system and method for blunt dissection of tissues and more specifically to a surgical device which can be used to blunt dissect pelvic floor tissues and provide rapid, safe and accurate access to the sacro-spineous ligament with minimal tissue trauma while also being capable of guiding the positioning of a tissue repair device and/or placing a mesh implant for reinforcing the damaged pelvic floor fascia with circumstances of pelvic organ prolapse and pelvic floor herniation and/or relaxation.

Surgical procedures necessitate dissection and displacement of tissue in order to provide access to anatomical landmarks and/or structures.

Oftentimes, tissue access is achieved via blunt tissue dissection. Blunt dissection generally allows for tissues to be dissected atraumatically by simply separating the tissue along existing natural planes. Blunt dissection provides both dissection and retraction while reducing tissue trauma.

One surgical procedure which utilizes blunt dissection is trans-vaginal pelvic floor repair. In such a procedure, blunt tissue dissection is utilized to provide access to the sacrospineous ligament from the posterior vaginal wall. A sling or mesh is then anchored to the sacrospineous ligament and the vaginal apex or the uterine isthmical fibrotic ring, cervix or body, to thereby support prolapsing tissues and/or organs.

Although pelvic floor repair is a common procedure, access to the sacrospineous ligament is typically effected by improvised manual blunt dissection techniques and/or use of off the shelf instruments. The sacrospineous ligament is a level 1 supportive fixation ligament and requires precise dissection to avoid ligament damage. As such, improvised dissection techniques often target a level 2 (inferior) supportive fixation points which require less tissue dissection with a reduced likelihood of ligament damage.

While reducing the present invention to practice, the present inventors have devised a surgical device which can be used to blunt dissect pelvic floor tissues and provide access to anatomical landmarks and structures such as the iscial spine and the sacro-spineous ligament while also being capable of guiding a tissue repair device and fixating it to target tissue.

### SUMMARY OF THE INVENTION

According to one aspect of the present invention there is provided a surgical device comprising an elongated device body having a proximal end and a distal end, a tissue dissecting head pivotally attached to the distal end and having at least one pair of movable jaws configured for blunt tissue dissection and an element for preventing trapping of tissue between the at least one pair of movable jaws.

According to further features in preferred embodiments of the invention described below, the element for preventing trapping of tissue between the at least one pair of movable jaws is an elastic sleeve covering the at least one pair of movable jaws. According to still further features in the described preferred embodiments the element for preventing trapping of tissue between the at least one pair of movable jaws is a finger-like extension positioned between the at least one pair of movable jaws.

According to still further features in the described preferred embodiments the surgical device further comprises an actuator attached to the proximal end of the elongated device body, the actuator being for opening and closing the at least one pair of movable jaws and for pivoting the tissue dissection head.

According to still further features in the described preferred embodiments the actuator includes a manual trigger for separately triggering the opening and closing of the at least one pair of movable jaws and for the pivoting of the tissue dissection head.

According to still further features in the described preferred embodiments the manual trigger is actuatable to a first position for opening and closing of the at least one pair of movable jaws and to a second position for pivoting of the tissue dissection head.

According to still further features in the described preferred embodiments the first and the second positions lie along a single actuation path.

According to still further features in the described preferred embodiments the elongated device body includes a longitudinal channel extending to the distal end of the elongated device body, wherein an opening of the channel at the distal end is exposed when the tissue dissection head is pivoted away from the elongated device body.

According to still further features in the described preferred embodiments the longitudinal channel includes a longitudinal slot through the elongated device body.

According to another aspect of the present invention there is provided a surgical device comprising an elongated device body having a proximal end and a distal end, a tissue dissecting head being attached to the distal end and having at least one pair of movable jaws configured for blunt tissue dissection and a longitudinal channel extending to the distal end of the elongated device body, wherein the longitudinal channel includes a longitudinal slot through the elongated device body.

According to still further features in the described preferred embodiments the surgical device further comprises an element for preventing trapping of tissue between the at least one pair of movable jaws.

According to still further features in the described preferred embodiments the element is an elastic sleeve covering the at least one pair of movable jaws.

According to still further features in the described preferred embodiments the element for preventing trapping of tissue between the at least one pair of movable jaws is positioned between the at least one pair of movable jaws.

According to still further features in the described preferred embodiments the tissue dissecting head is pivotally attached to the distal end.

According to still further features in the described preferred embodiments the surgical device further comprises an actuator attached to the proximal end of the elongated device body, the actuator being for opening and closing the at least one pair of movable jaws and for pivoting the tissue dissection head.

According to still further features in the described preferred embodiments the actuator includes a manual trigger for separately triggering the opening and closing of the at least one pair of movable jaws and for the pivoting of the tissue dissection head.

According to still further features in the described preferred embodiments the manual trigger is actuatable to a first position for opening and closing of the at least one pair of movable jaws and to a second position for pivoting of the tissue dissection head.

According to still further features in the described preferred embodiments the first and the second positions lie along a single actuation path.

According to still further features in the described preferred embodiments an opening of the channel at the distal end is exposed when the tissue dissection head is pivoted away from the elongated device body.

According to yet another aspect of the present invention there is provided system for pelvic floor repair comprising the surgical device described above and a suture attached to a tissue anchor.

According to still further features in the described preferred embodiments the system further comprises a mesh attached to the suture.

According to still further features in the described preferred embodiments the elongated device body is curved along at least a portion of its length.

According to still further features in the described preferred embodiments the radius of curvature of the elongated device body is 9-11 cm along a sector of 45 degrees.

According to still another aspect of the present invention there is provided a method of providing tissue access to an anatomical landmark comprising: (a) providing a surgical device including: (i) an elongated device body having a proximal end and a distal end; and (ii) a tissue dissecting head being attached to the distal end (optionally via a pivoting link) and having at least one pair of movable jaws configured for blunt tissue dissection; (b) actuating the at least one pair of movable jaws of the surgical device to blunt dissect tissue in a first path; and (c) actuating the at least one pair of movable jaws of the surgical device while pivoting the dissection head with respect to the elongated device body to blunt dissect tissue in a second path thereby providing tissue access to the anatomical landmark.

According to still further features in the described preferred embodiments the anatomical landmark is a sacrospineous ligament.

According to still further features in the described preferred embodiments the first path is from a posterior vaginal wall.

According to still further features in the described preferred embodiments the second path and the first path are in the same dissection plane.

According to still further features in the described preferred embodiments the first path is from the posterior vaginal wall in a direction of an ischial spine.

According to still further features in the described preferred embodiments the second path is from an ischial spine region in a direction of the sacrospineous ligament.

According to still another aspect of the present invention there is provided method of repairing a pelvic floor disorder comprising: (a) providing a surgical device including: (i) an elongated device body having a proximal end and a distal end; (ii) a tissue dissecting head being attached to the distal end (optionally via a pivoting link) and having at least one pair of movable jaws configured for blunt tissue dissection; and (iii) a longitudinal channel extending to the distal end of the elongated device body; (b) actuating the at least one pair of movable jaws of the surgical device to blunt dissect tissue in a first path; (c) actuating the at least one pair of movable jaws of the surgical device while pivoting the dissection head with respect to the elongated device body to blunt dissect tissue in a second path thereby providing tissue access to the anatomical landmark; (d) pivoting the tissue dissecting head away from the elongated device body to thereby expose a distal opening of the longitudinal channel; (e) using the longitudinal channel to deliver a tissue repair device to the anatomical landmark thereby repairing the pelvic floor disorder.

According to still further features in the described preferred embodiments the pelvic floor disorder is central pelvic apical prolapse.

The present invention successfully addresses the shortcomings of the presently known configurations by providing a surgical device that can be used for blunt dissection of tissues and guiding of a tissue repair device to an anatomical landmarks and structures through the dissected tissue path.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates an embodiment of the present surgical device showing the elongated device body and attached tissue dissecting head.

FIG. 2 illustrates the surgical device of FIG. 1 with the pair of movable jaws in the open position.

FIG. 3 illustrates the surgical device of FIG. 1 with the tissue dissecting head pivoted with respect to a longitudinal axis of the elongated device body.

FIG. 4 illustrates the surgical device of FIG. 1 with the pair of movable jaws in the closed position and covered with an elastic sleeve.

FIG. 5 illustrates the surgical device of FIG. 1 with the pair of movable jaws in the open position and covered with an elastic sleeve.

FIG. 6 illustrates an embodiment of the present surgical device showing the elongated device body with attached tissue dissecting head and actuating handle.

FIG. 7 illustrates an embodiment of the present surgical device showing the elongated device body with attached tissue dissecting head and a channel extending to a distal end of the elongated device body.

FIG. 8 illustrates an embodiment of the present surgical device showing the elongated device body with attached tissue dissecting head and a slotted channel extending to a distal end of the elongated device body.

FIG. 9A is an exploded view of the jaws of the device illustrated in FIG. 6.

FIG. 9B is a cutaway view of the handle of the device of the present invention.

FIGS. 10A-C illustrate mesh deployment using the device of the present invention.

FIG. 11 illustrate the dissection path of the present surgical device in repair of a pelvic floor disorder.

FIG. 12 illustrates another embodiment of the present surgical device showing a curved elongated device body and attached tissue dissecting head.

FIG. 13 illustrates the device of FIG. 12 with the pair of movable jaws in the open position showing the finger like extension for preventing tissue trapping.

FIGS. 14-16 illustrate mesh guiding and deployment using the device of FIG. 12.

FIGS. 17A-C illustrate the movement path of the dissecting head jaws of the device of FIG. 12.

FIG. 18 illustrates distance markers useful for determining a depth of tissue penetration of the device body.

### DESCRIPTION OF THE PREFERRED EMBODIMENTS

The present invention is of a surgical device which can be used to generate a blunt dissection tissue path and provide access to anatomical landmarks. Specifically, the present invention can be used to access pelvic floor tissue and guide positioning of tissue repair devices such as sutures, slings and/or meshes used to repair pelvic floor prolapse.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description, Example or drawings. The invention is capable of other embodiments or of being practiced or carried out in various ways. Also, it is to be understood that the phraseology and terminology employed herein is for the purpose of description and should not be regarded as limiting.

Blunt dissection of tissue can provide access to anatomical landmarks with minimal tissue trauma. Although devices that can be used to blunt dissect tissue are common (e.g. blunt dissection forceps), such devices are designed for basic blunt dissection operability and as such, their functionality and adaptability are limited.

In order to overcome the limitations of prior art blunt dissectors, the present inventors have devised a surgical device that can provide a solution for surgical approaches that require blunt dissection of tissue along several paths, while minimizing tissue trauma. As is further described herein, the present device is also configured for viewing an anatomical landmark and guiding a tissue repair device thereto.

Thus, according to one aspect of the present invention, there is provided a surgical device which can be used for blunt dissection of tissues, such as, for example, muscle, fat, connective tissue and the like of a treated subject such as a human.

The present device includes an elongated body which is attached to a tissue dissecting head having a tissue biasing mechanism including one or more movable elements capable of blunt dissecting tissues along one or more dissection planes. The tissue dissecting head is preferably pivotally attached to a distal end of the elongated device body and is thus capable of angulation with respect to a longitudinal axis of the device body. The movable elements of the tissue dissecting head are preferably configured as movable jaws which when opened, bias surrounding tissues outwardly thus producing a tissue separation force. The movable jaws include a mechanism (e.g. elastic sleeve cover) for preventing trapping of tissue within the jaws.

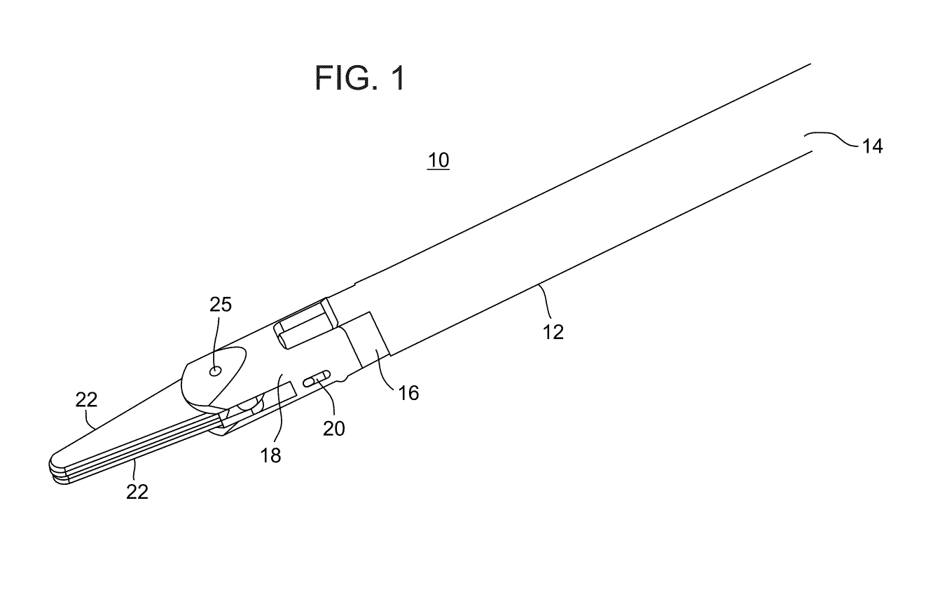

FIGS. 1-9 illustrate embodiments of the present device which is referred to herein as device 10.

Device 10 includes an elongated device body 12 having a proximal end 14 and a distal end 16. Elongated body (6-10 cm in length) enables access of distal end 16 to the sacro-spineous ligament. The diameter of elongated device body 12 is typically less than that of an index finger (about 1-1.5 cm) to avoid tissue trauma.

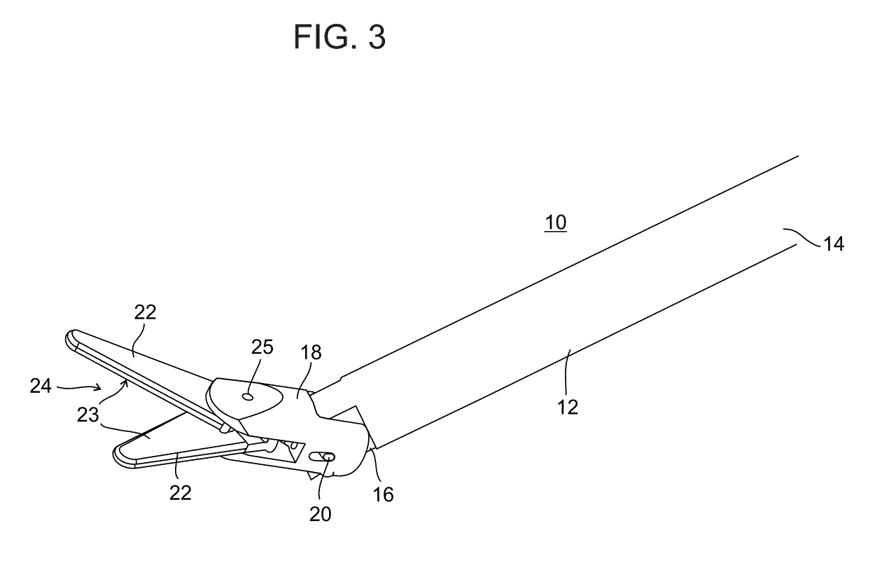

Device 10 further includes a tissue dissecting head 18 which is preferably attached to distal end 16 via a pivot 20. Pivot 20 can enable rotation of dissecting head 18 through a single axis (e.g. swivel joint or a hinge joint) or it can enable rotation around an infinite number of axis (e.g. ball and socket); FIG. 3 illustrates angulation of dissecting head 18 around a hinge-type pivot 20. Dissecting head 18 can also be capable of additional/alternative movement (e.g. heaving, swaying and surging) with respect to distal end 16 of elongated device body 12.

Dissecting head 18 includes tissue biasing element(s), which are preferably configured as one or more pair(s) of movable jaws 22 (one pair shown) which pivot in a scissor-like action around pin 25. Jaws 22 are configured for blunt tissue dissection and as such are flat and blunt. Jaws 22 can be made out of any metal or alloy (e.g. stainless steel, titanium) or any other rigid material.

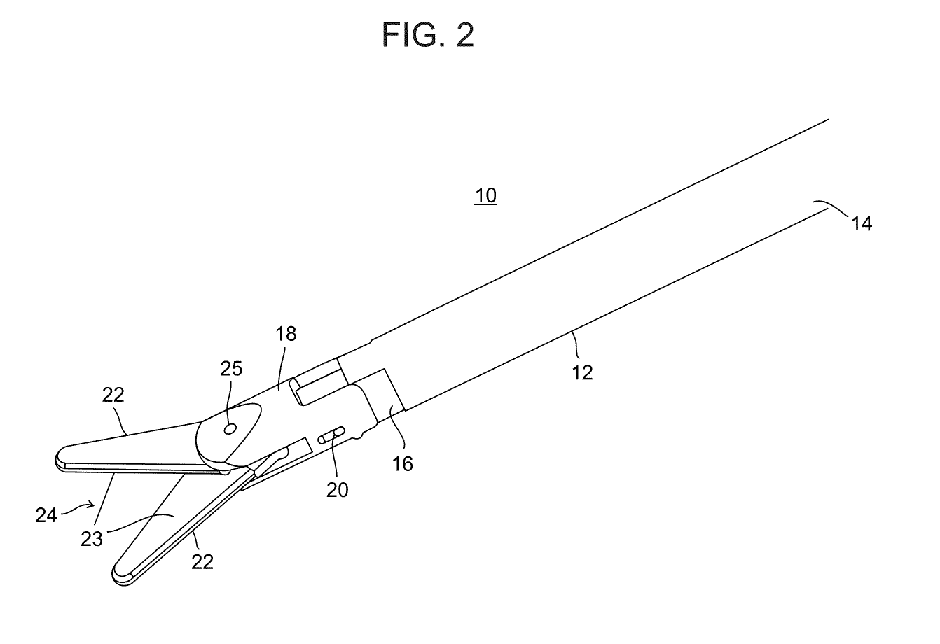

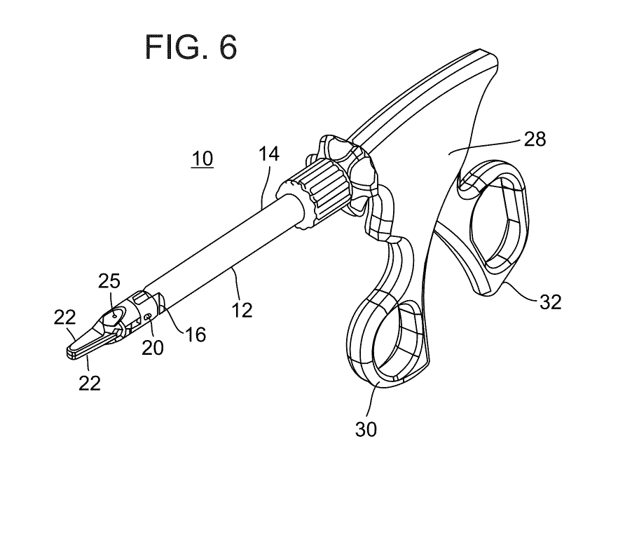

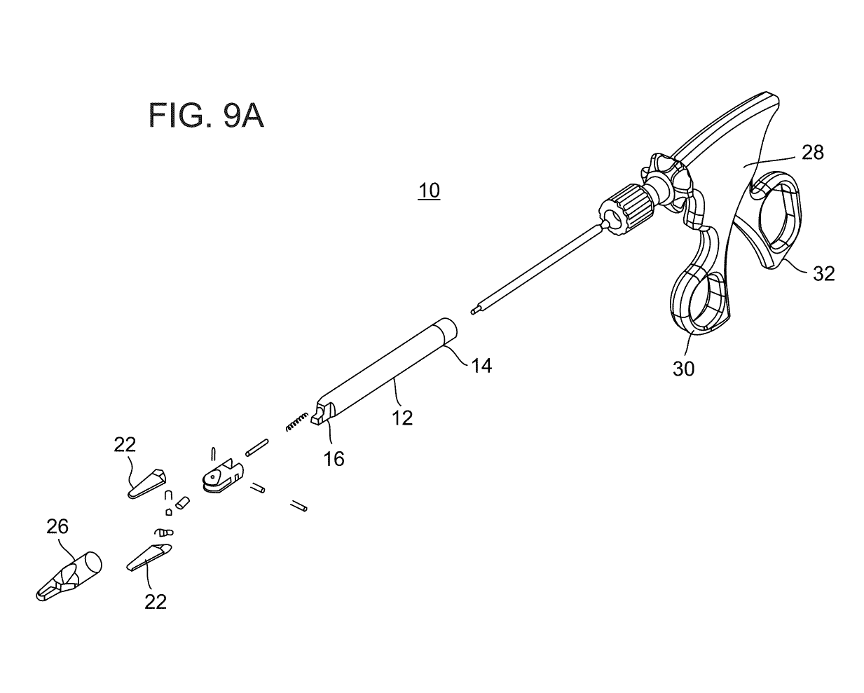

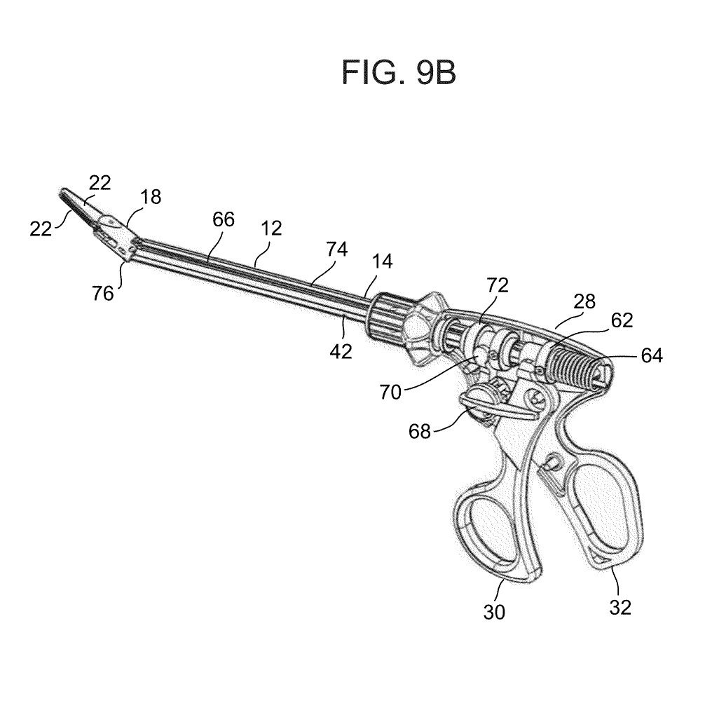

Jaws 22 are actuatable between closed (FIG. 1) and open (FIG. 2) positions (and anywhere in between) via a manual or motorized actuating mechanism (further described hereinbelow with respect to FIGS. 6 and 9).

In order to prevent trapping/pinching of tissue within jaws 22 during blunt dissection, device 10 further includes a mechanism for preventing tissue from occupying a space 24 (FIG. 2) between jaws 22 when in an open position. Such a mechanism can be a non-movable flat finger-like extension (not shown) that interposes between inside surfaces 23 (FIG. 2) of jaws 22 when closed, and occupies space 24 when jaws 22 are opened.

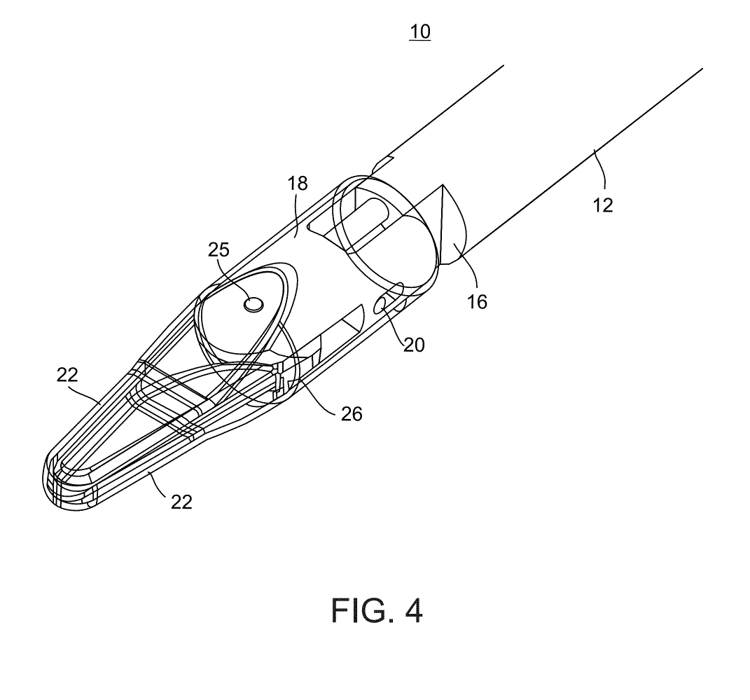

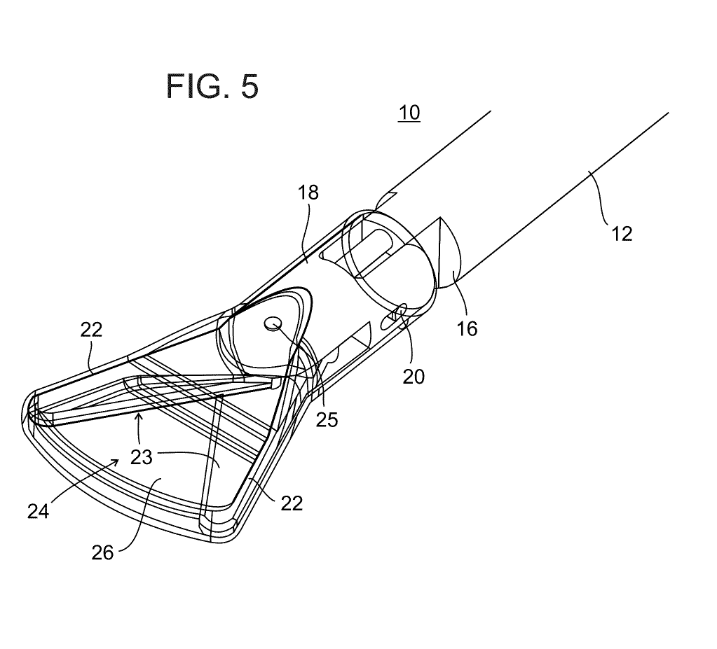

Alternatively, and as shown in FIGS. 4 and 5, device 10 can include an elastic cover 26 around dissecting head 18. Cover 26 can be fabricated from silicon, latex or the like and be capable of elastically stretching when Jaws 22 are opened (FIG. 5). Cover 26 can be shaped as a closed end sleeve 10 mm or less in diameter and having an elastic modulus of less than 50 shore.

As shown in FIG. 6, device 10 also includes an actuator 28 for actuating movement of dissecting head 18 and of jaws 22. Actuation can be manual (as shown in FIG. 6) or motorized. A manually operated actuator preferably includes a pair of handles 30 and 32 which are operated via approximation or retraction thereof. Further description of the operation and internal mechanism of device 10 is provided with respect to FIG. 9 below.

Actuator can further include a selector 34 for selecting a mode of operation of handles 30 and 32 (opening and closing of jaws 22 and/or pivoting of dissection head 18). Selector 34 indicates the angle of dissection head 18. When selector 34 is parallel to elongated device body 12, dissection head 18 is straight (i.e. aligned with device body 12). When selector 34 is not parallel to elongated device body 12, dissection head 18 is at an angle (i.e. not aligned with device body 12).

Selector 34 as well as handle 30 are ergonomically designed to enable a single finger operation forces.

Selector 34 can be positioned in two or more positions along its path, enabling partial movement of dissection head 18. The virtual plane created between jaws 22 indicates the dissection plane.

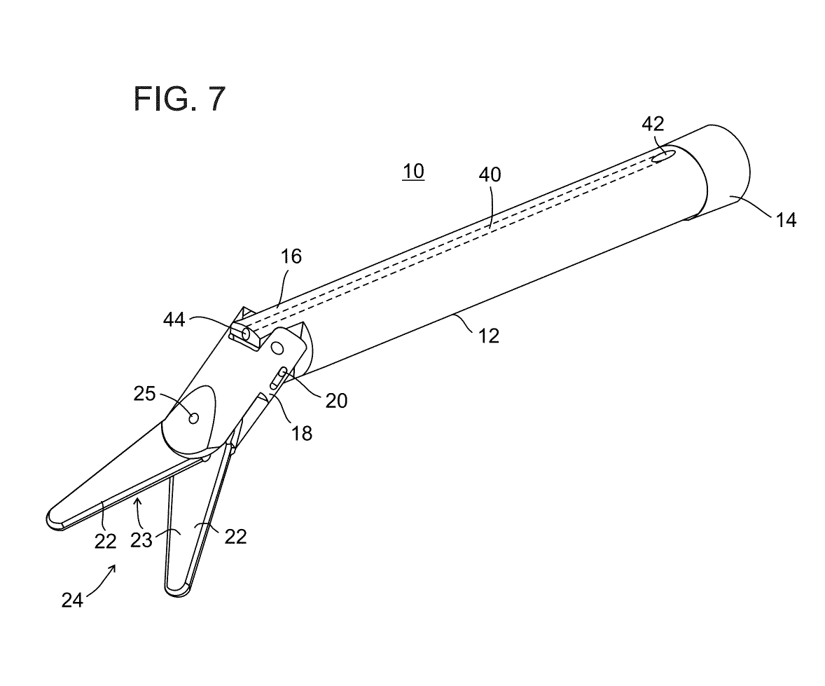

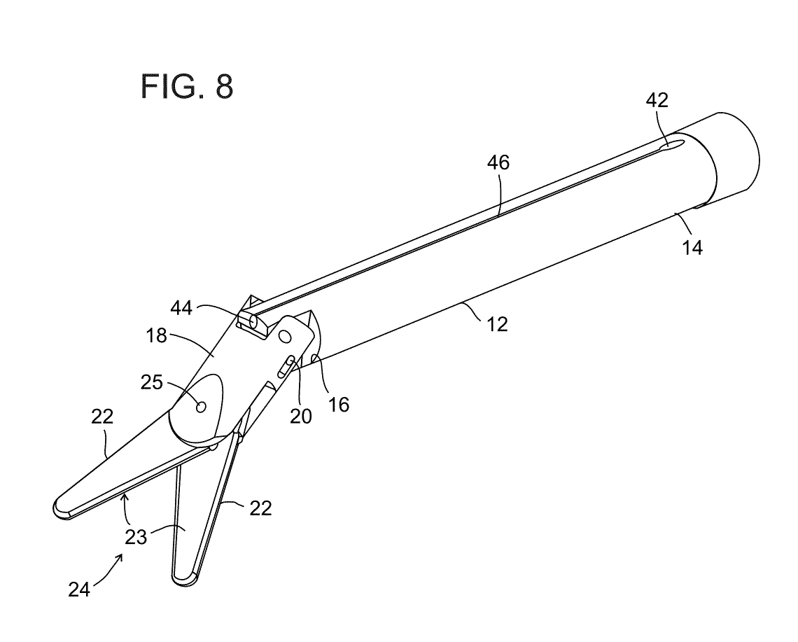

As shown in FIGS. 7-8 device 10 further includes a channel 40 (about 2 mm in diameter) running a length of elongated device body 12 and having a proximal opening 42 at proximal end 14 and a distal opening 44 at distal end 16. Channel 40 can include a ˜0.6mm slot 46 running a length thereof. Slot 46 can be used to release a device (e.g. suture) disposed within channel 40 without having to remove device 10 from the body. Channel 40 having a slot 46 can further include an internal sleeve (fabricated from an alloy or a polymer) running a length of channel 40. Such a sleeve (not shown) can be slotted along its length and be rotated within channel 40. The sleeve can be used to prevent tissue invagination into channel 40 (through slot 46) or unwanted release of a device such as a suture from channel 40 (through slot 46). Thus, when used to guide a device to a target tissue, slot 46 of channel 40 and the slot of the internal sleeve can be misaligned and when release of the channel-positioned device is desired an operator simply aligns the slots to provide a release pathway.

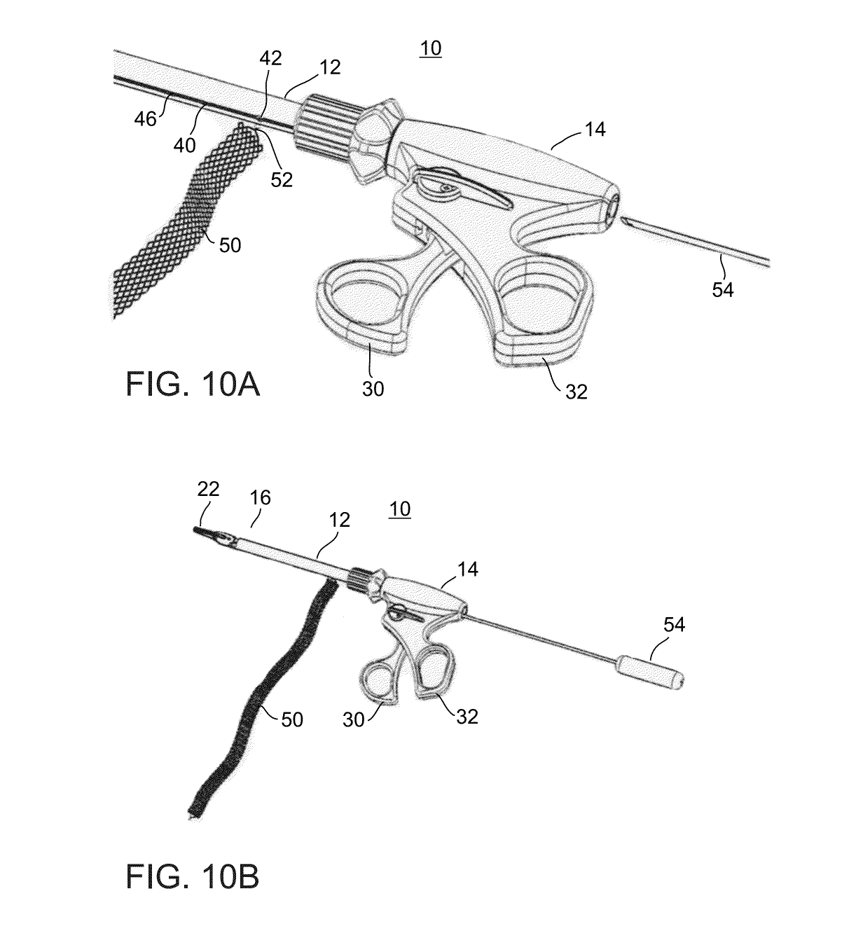

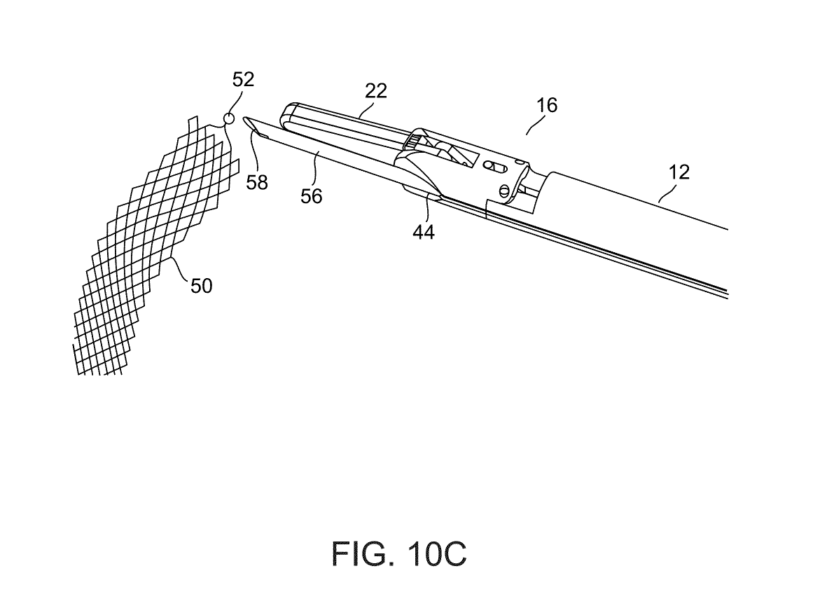

Channel 40 and slot 46 can be used to guide a mesh or sling to a target tissue. FIGS. 10a-c illustrate a mesh 50 which is provided with an element that can be inserted into channel 40 through proximal opening 42. Element 52 can be a suture knot, an alloy sphere or any other element that can be trapped within the internal lumen of channel 40 and translated therethrough (FIG. 10b) using, for example, a push rod 54 which is inserted from a proximal end 14 of device 10. Once element 52 reaches distal end 16 it can be released from channel 40 at distal opening 44. In order to enable close approximation of mesh 50 to the target tissue, push rod 54 can be provided with a distal portion 56 having a beveled tip 58 which can be extended out of distal opening 44 of channel 40 to contact and optionally pierce the target tissue.

Channel 40 can also be used to deliver drugs, for aspiration, imaging, to deliver a blood clot to target tissue and the like.

FIG. 9a illustrates link 60 that transfers movement of handle 30 to jaws 22. As handle 32 is pulled towards the proximal end of the device, the jaws spread open.

FIG. 9b illustrates the internal parts of device 10. Actuator 28 includes two independent link mechanisms; each controlled separately. Movement of handle 30 is transferred via sliding member 62—which is biased by spring 64 and is connected to link 66—to jaws 22 at distal end of the device. As handle 30 is pushed distally jaws 22 spread open.

Articulation levers 68 (one shown) positioned on both sides of device 10 (for left or right handed operation) transfers movement through arm 70 that pushes sliding member 72. Sliding member 72 is connected to link 74 that pivots head 16 around pivot pin 76 in order to change the angle of dissection head 18.

A CMOS or a CMOS-like device can be connected and attached to the distal end of the present device to allow intrabody imaging of tissues, in addition an RF tip (blunt needle) can be incorporated into the device for ablation purposes or/and any of the shelf RF device.

Device 10 of the present invention can be used to blunt dissect any tissue of any organ or body region. As such, device 10 of the present invention can be used in surgical procedures such as anterior prolapse dissection, male anti-incontinence surgery, orthopedic reconstruction of torn ligaments, cosmetic surgery and the like.

One presently preferred use of device 10 of the present invention is in pelvic floor prolapse repair procedures and specifically in blunt dissecting a tissue pathway to a sacrospineous ligament and in addition, guiding a suture anchor and optionally mesh thereto.

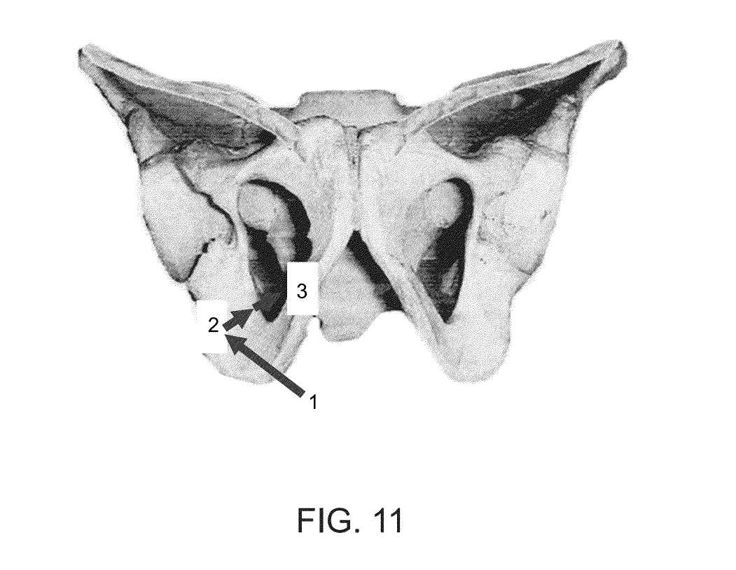

FIG. 11 illustrates a dissection path of the present device in generating a tissue pathway to a sacrospineous ligament. Briefly, the posterior vaginal wall is uncovered and a saline mixture which includes 100 ml of saline and local anesthetic (e.g., xylocaine 1%) +/−adrenalin 0.2 ml (1000 units/ml) is administered locally via 20 ml injections 2-4 mm into a region 2-3 cm distal to the posterior cervix or vaginal apex. An incision 2-3 cm distal to the cervix or vaginal apex is made at the site of infiltration and the incision is expanded up to 1.5 cm towards the perineum once the correct layer under the fascia is identified. The edges of the vaginal incision are held apart using two Allis clamps and the vaginal wall and underlying fascia are retracted. A 1-2 cm incision is made to create an entry point for device 10. Dissection using device 10 starts from midline, below the vaginal wall and underlying fascia, through the areolar tissue toward the pelvic side wall (sub-obturator region) a distance of 7.5 cm (arrow 1, FIG. 11). Dissection then proceeds for 2 cm along the same plane in a direction towards the ischial spine (arrow 2, FIG. 11) and then an additional 2 cm at the same direction toward the sacro-spineous ligament (arrow 3, FIG. 11). This positions the distal end of device 10 about 1 cm above the central portion of the sacro-spineous ligament.

A mesh can then be guided along channel 40 (as described above) and anchored in the sacro-spineous ligament using anchors sutures or the like.

As is described in Example 2 of the Examples section which follows, studies utilizing the blunt dissector of the present invention have provided valuable insight as to the operation of device 10. Specifically, the present inventors have discovered that in pelvic floor procedures introduction of device 10 at the right plane and angle can be limited by space limitations and presence of arteries and nerves such as the pudental bundle.

Such insights guided the present inventors in development of a curved blunt dissector which is optimized for blunt dissection of pelvic floor tissues as well as other tissues which impose navigational constraints.

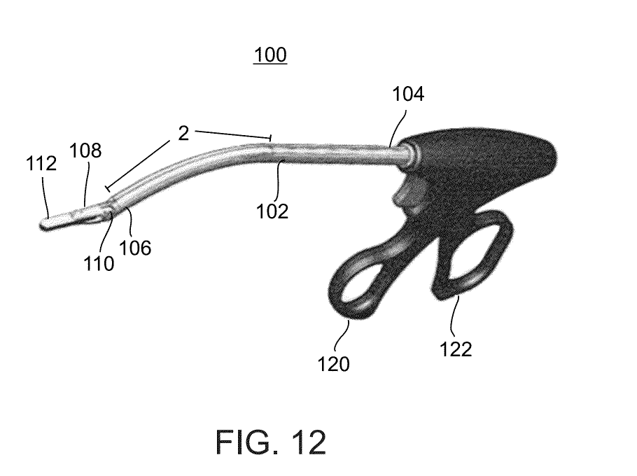

FIGS. 12-16 illustrates the curved blunt dissector device of the present invention which is referred to herein as device 100.

Device 100 includes an elongated device body 102 which is generally cylindrical/oval in cross section and is fabricated from an alloy (e.g. stainless steel, titanium) or polymer (e.g. re-enforced glass fiber, carbon fiber, Nylon, ABS and the like). Elongated device body 102 has a proximal end 104 and a distal end 106 and a length of 5-25 cm which enables access of distal end 106 to the sacrospineous ligament when the handle of device 100 is positioned outside the dissected tissue path (e.g. outside the body). The diameter of elongated device body 102 is typically less than that of an index finger (about 1-1.5 cm) to avoid tissue trauma.

Device body 102 is curved along at least a portion of its length (curved portion designated as L in FIG. 12). The curvature of device body 102 follows a radius of curvature of 9-11 cm, preferably 10 cm along a sector of about 45 degrees (equates to about 7-10 cm of curvature or 50% of a total length of device body 102).

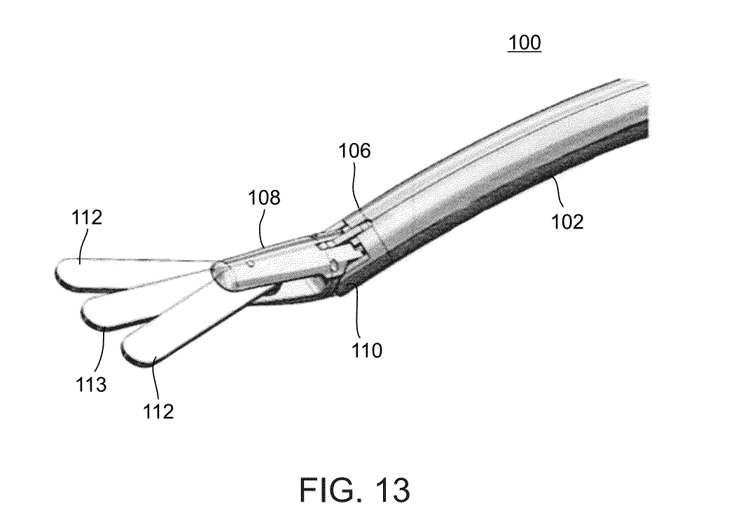

Device 100 further includes a tissue dissecting head 108 which can be immovably attached to distal end 106, or is preferably attached thereto via a pivot 110. Pivot 110 can enable rotation of dissecting head 108 through a single axis (e.g. swivel joint or a hinge joint) or it can enable rotation around an infinite number of axis (e.g. ball and socket); FIG. 13 illustrates angulation of dissecting head 108 around a hinge-type pivot 110. Dissecting head 108 can also be capable of additional/alternative movement (e.g. heaving, swaying and surging) with respect to distal end 106 of elongated device body 102. This angulation enables device 100 to navigate towards the desired tissue target while staying close to the pelvic side-wall and away from organs as intestines, bladder, ureters, blood vessels, nerves etc.

Dissecting head 108 preferably swivels via a hinge joint along the plane of curvature of elongated device body 102. i.e. the plane of swivel is the same plane of curvature. As is further described in Example 2 of the Examples section which follows this enables articulation of dissecting head 108 away from (opposite to) the curvature of elongated device body 102 and thus enables dissection in a direction other then the axis of shaft 102.

Dissecting head 108 can pivot continuously through a range of pivoting motion (+/−90 degrees with respect to distal end 106) or it can be actuated to through predefined pivoting steps. For example, dissecting head 108 can be indexed to move through two articulation steps, a first which angles dissecting head 108 at 15 degrees (with respect to distal end 106) and second to which pivots dissecting head 108 at 30 degrees (with respect to distal end 106).

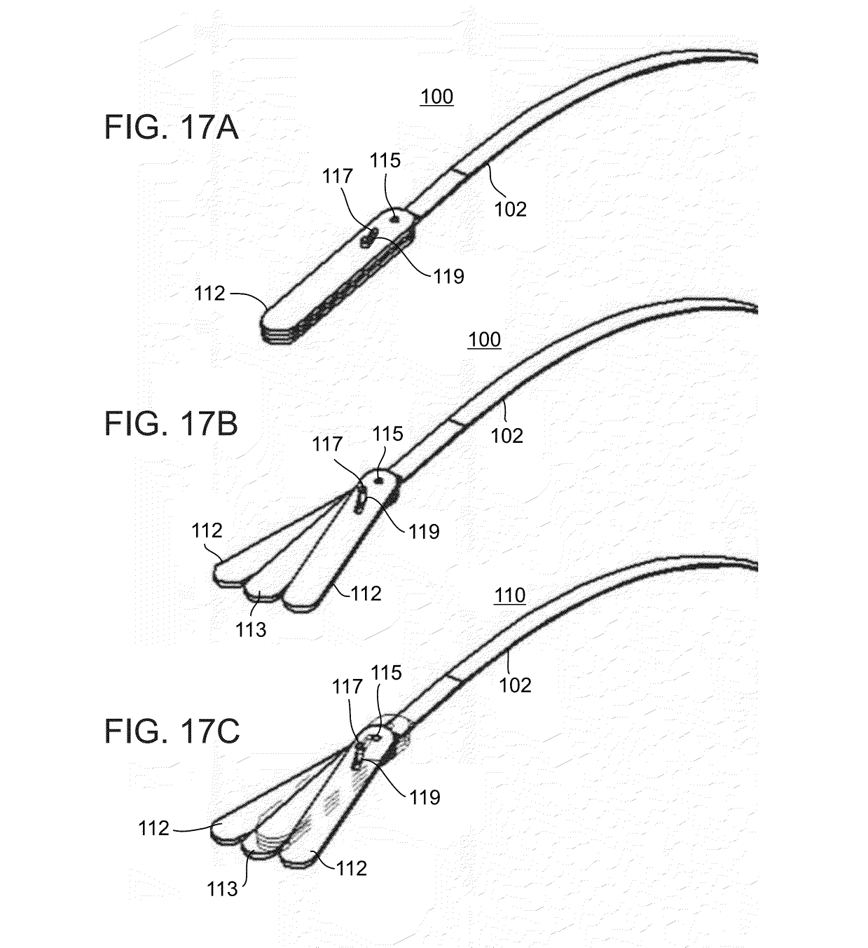

Dissecting head 108 includes tissue biasing element(s), which are preferably configured as one or more pair(s) of movable jaws 112 (one pair shown). Jaws 112 follow open-out-close-in path where movable jaws 112 first move sideways (open), then out (in a distal direction—away from device body 102), then close and move back in (in a proximal direction—towards device body 102). This path of movement which is illustrated in FIGS. 17a-c, enhances the ability of movable jaws 112 to blunt dissect and separate the tissues.

Jaws 112 are configured for blunt tissue dissection and as such are flat and preferably blunt. Each Jaw 112 is configured as a flat alloy (titanium or stainless steel) having a length of 2-4 cm, a width of 3-8 mm and a thickness of 0.3-1.5 mm. Each jaw 112 is separately attached to a floating hinge 115 (the right one on the new figures). A pin 117 is fixed to head 108. Pin 117 along with jaws 112 is actuates by a pusher mechanism which is actuated by the main handle. A groove 119 in each of jaws 112 guides jaws 112 through the movement path.

Jaws 112 are actuatable between closed (FIG. 12) and open (FIG. 13) positions (and anywhere in between) via a manual or motorized actuating mechanism (further described hereinbelow).

In order to prevent trapping/pinching of tissue within jaws 112 during blunt dissection, device 100 further includes a mechanism for preventing tissue from occupying a space between jaws 112 when in an open position. Such a mechanism is preferably a flat finger-like extension 113 (FIG. 13) which is similar in shape to jaw 112. Extension 113 is interposed between jaws 112 when closed and occupies a space in between jaws 22 when open.

Device 100 also includes an actuator for actuating movement of dissecting head 108 and of jaws 112. Actuation can be manual or motorized. A manually operated actuator preferably includes a pair of handles 120 and 122 (FIG. 12) which are operated via approximation or retraction thereof. The actuator of device 100 is similar in structure and function to actuator 28 described above for device 10.

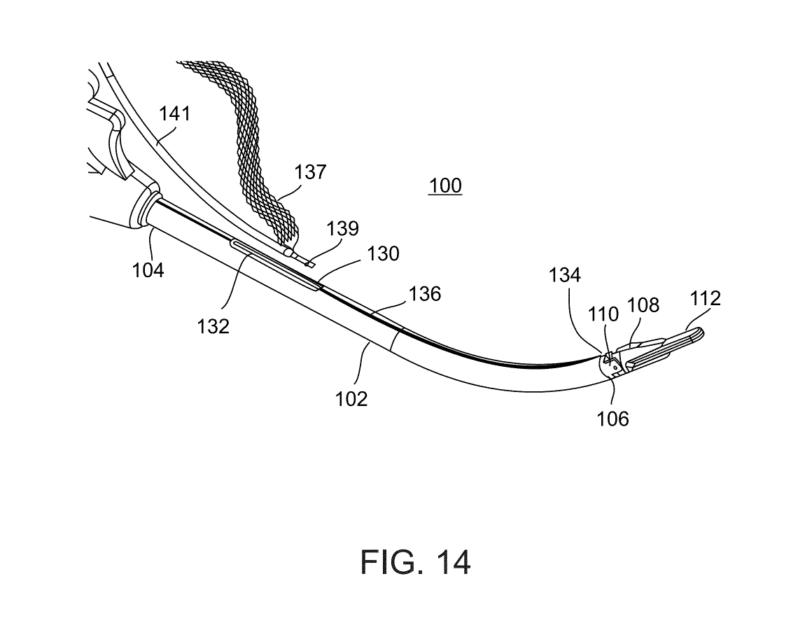

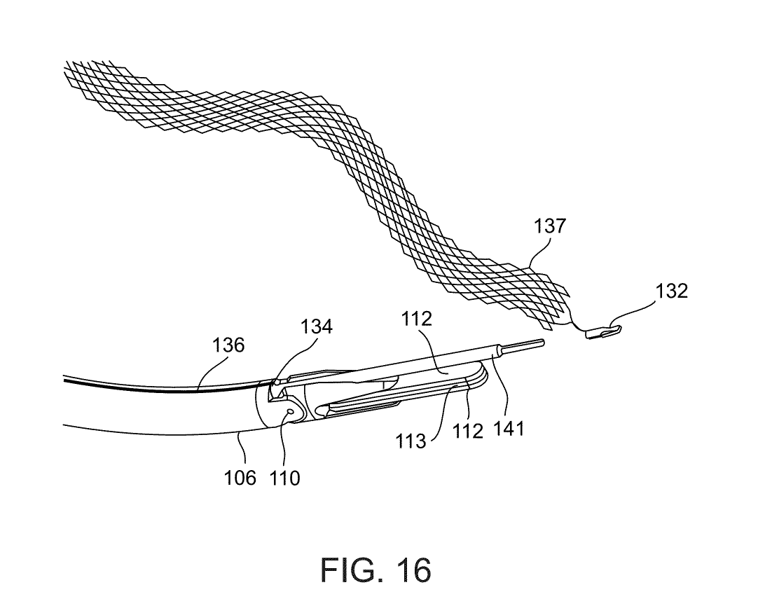

As is shown in FIG. 14, device 100 further includes a channel 130 (about 2-4 mm in diameter) running a length of elongated device body 102 and having a proximal opening 132 at proximal end 104 and a distal opening 134 at distal end 106. Distal opening 134 can open over or under dissecting head 108 (as is shown in FIG. 16), or alternatively it can be closed (by dissecting head 108 or jaws/extension 112-113) and exposed (opened) when dissecting head 108 is pivoted away or when jaws 112 (and extension 113) of dissecting head 108 are opened, or are angled away from the center axis.

Channel 130 preferably includes a 1.0 mm slot 136 running a length thereof. Slot 136 can be used to load and release a tissue repair device (e.g. mesh 137) through channel 130 without having to remove device 100 from the body. This is particularly useful in cases where device 100 blunt dissects a path to a tissue targeted for repair or manipulation (e.g. sacrospineous ligament or ischial bone).

Channel 130 can further include an internal sleeve (fabricated from an alloy or a polymer). Such a sleeve (not shown) can be slotted along its length and be rotated within channel 130 to prevent tissue invagination into channel 130 (through slot 136) or unwanted release of a tissue repair device (such as a suture) from channel 130 (through slot 136). Thus, when used to guide a tissue repair device to a target tissue, slot 136 of channel 130 and the slot of the internal sleeve can be misaligned and when release of the channel-positioned device is desired an operator simply aligns the slots to provide a release pathway.

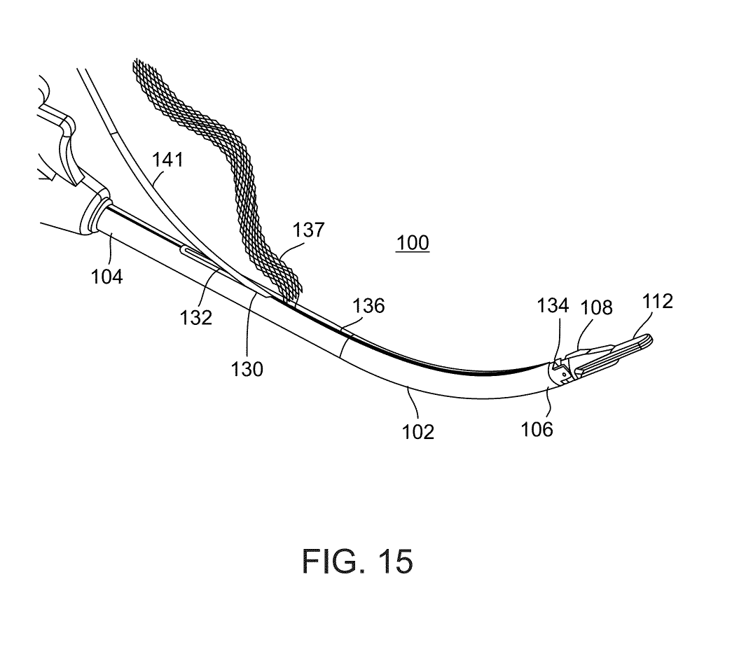

Channel 130 and slot 136 can be used to guide a mesh 137 to a target tissue. FIGS. 14-16 illustrate a mesh 137 which is provided with an element 139 (shuttle) that can be mounted on a guide 141 and inserted therewith into the internal lumen of channel 130. Guide 141 can then be used to push element 139 (and attached mesh 137) through channel 130 (FIG. 15) and out through distal opening 134 (FIG. 16).

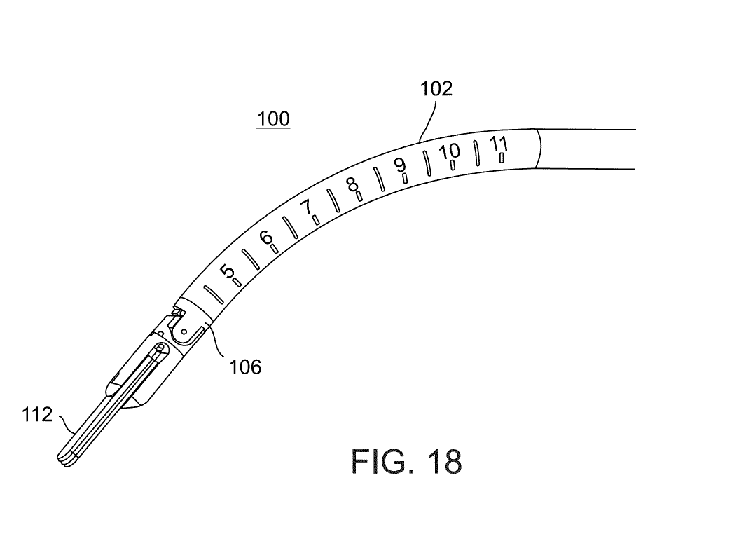

To enhance guidance through the tissue path and facilitate correct targeting to anatomical landmarks, device 100 further includes distance markers on device body 102 (see FIG. 18).

Device 100 can be used to blunt dissect a path through any tissue of the body. As is further described in Example 2 of the Examples section which follows, device 100 is particularly useful in blunt dissection and repair associated with pelvic floor disorders.

For example, in an anterior apical support procedure, device 100 is introduced through an incision in the vaginal wall and used to blunt dissect a path towards the para-vesical area, at a plane parallel to the vaginal wall. Dissection is then directed towards the ischial tuberosity at a 30 degree angle to the vaginal wall plane. At a depth of about 7.5 cm (as determined by the distance markers), device 100 is at the pelvic side wall just below the obturator foramen. The surgeon maintains an index finger inside the vagina, feeling the spine at all time in order to ensure that device 100 passes over the spine. Dissection head 108 is then articulated medially and the dissection jaws are used to blunt dissect an additional 2 cm at the same plane along the pelvic side wall in the direction of the ischial spine. Device 100 is then used to blunt dissect an additional 2 cm toward the MSSL. When dissection is complete dissection head 108 is at the MSSL or 1 cm above it. Throughout the procedure, channel 130 remains visible to the surgeon.

Following dissection, an anchor (with attached mesh or suture) is connected to guide 141 which is inserted into channel 130. Guide 141 is advanced along channel 130 until the anchor contacts and fixates to the MSSL. The attached suture or mesh can then be released through slot 136 and Device 100 removed from the body.

Device 100 can also be used in a cystocele procedure by introducing it through an incision in the vaginal wall and using it to blunt dissect a path towards the para-vesical area, at a plane parallel to the vaginal wall. Dissection is then directed towards the ischial tuberosity at a 30 degree angle to the vaginal wall plane. At a depth of about 7.5 cm (as determined by the distance markers), device 100 reaches the pelvic side wall at the obturator and/or ATFP region.

Following dissection, an anchor (with attached mesh or suture) is connected to guide 141 which is inserted into channel 130. Guide 141 is advanced along channel 130 until the anchor contacts and fixates to the soft tissue of the obturator region and/or ATFP. The attached suture or mesh can then be released through slot 136 and Device 100 removed from the body.

Device 100 can also be used in an enterocele/rectocele procedure by introducing it through an incision in the vaginal wall and using it to blunt dissect a path towards the para-vesical area, at a plane parallel to the vaginal wall. Dissection is then directed towards the ischial tuberosity at a 30 degree angle to the vaginal wall plane. At a depth of about 7.5 cm (as determined by the distance markers), device 100 reaches the pelvic side wall at the obturator foramen.

Following dissection, an anchor (with attached mesh or suture) is connected to guide 141 which is inserted into channel 130. Guide 141 is advanced along channel 130 until the anchor contacts and fixates to the soft tissue of the pelvic side-wall. The attached suture or mesh can then be released through slot 136 and Device 100 removed from the body.

Device 100 can also be used in a vaginal sacrocolpopexy/hysteropexy procedure. Device 100 is introduced through an incision in the vaginal wall and used to blunt dissect a path towards the pararectal area and ischio-rectal space, at the plane parallel to the virginal wall. Dissection is then directed towards the ischial tuberosity at a 30 degree angle to the vaginal wall plane. At a depth of about 7.5 cm (as determined by the distance markers), device 100 is at the pelvic side wall just below the obturator foramen. The surgeon maintains an index finger inside the vagina, feeling the spine at all time in order to ensure that device 100 passes over the spine. Dissection head 108 is then articulated medially and the dissection jaws are used to blunt dissect an additional 2 cm at the same plane along the pelvic side wall in the direction of the ischial spine. Device 100 is then used to blunt dissect an additional 5 cm at same direction toward the sacrum, at the level of S-3, passing over the MSSL. When dissection is complete dissection head 106 is positioned at the sacrum. Dissection head 106 is then straightened and a screw is loaded onto guide 141 which is advanced through channel 130 and used to deliver the screw into the sacrum.

The present invention can also be a part of a system used in repair of pelvic floor disorders. Such a system can include device 10 or 100 and a tissue repair/treatment device such as a suture-anchor combination with or without an attached mesh or sling.

Fixation of the mesh to the soft tissue can be achieved by suturing using a suture and needle or a suturing device, by anchoring with a harpoon, by tuckers or by any other combinations thereof. The suture or anchors can be long standing or absorbable. The mesh can be made of any synthetic, biologic or organic material, long standing or absorbable, plain, knighted or woven, placed for reinforcing the native tissue contributing to the herniation/prolapse/relaxation of the pelvic floor.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Cadaver Study With the Non-Curved Blunt Dissector

A cadaver study was used to test the efficacy of the non-curved configuration of the present device in generating a tissue pathway to a Sacro-spineous ligament. The posterior vaginal wall was visualized and 20 ml of saline were injected at a depth of 2-4 mm. An incision was made 2-3 cm distal to the cervix or vaginal apex at the site of infiltration and the incision was extended up to 1.5 cm towards the perineum. The edges of the vaginal incision were held apart using two Babcock or Alice clamps and the vaginal wall was retracted to identify the fascia. The present device was introduced through the incision and was pointed towards the para-rectal area and ischio-rectal space. Blunt dissection was directed towards the Ischial Tuberosity with a 30 degree angle below the horizontal plane. Dissection proceeded along this plane for 7.5 cm until the pelvic side wall was reached as verified by manual inspection.

The device was then used to dissect an additional 2-3 cm at the same plane, along the pelvic side wall in the direction of the ischial spine (the ischial spine was reached as confirmed by manual inspection). Tissue was then dissected at same direction for about 2-3 cm to the sacro-spineous ligament (confirmed via manual inspection).

The formed tissue pathway (from entry point to Sacro-Spineous ligament) was then confirmed via manual inspection and a mesh was guided along the device channel and positioned using tweezers.

Laparotomy was then used to evaluate the accuracy of the dissection and mesh placement. The tissue pathway created by the present device and placement of the mesh as facilitated by the present device were as expected with the mesh being positioned 1 cm above the sacro-spineous ligament.

### Example 2

### Cadaver Study With Curved Blunt Dissector

A study was conducted in order to evaluate the suitability of the present device as a surgical tool in repair of pelvic floor disorders.

The study was conducted using five female cadavers; two transvaginal procedures were performed for each cadaver and the results were evaluated for mesh placement as well as suture placement for vaginal Colposacrospinopexy

The study evaluated the performance of the present device according to the following parameters:

(i) blunt dissection capabilities;

(ii) navigation capabilities (navigate to the pelvic side wall then to the Ischial spine and then to the MSSL).

(iii) dual-sided dissection (left and right)—starting at the high posterior virginal wall and passing through the posterior pelvic floor compartment; 2 dissections in each cadaver.

(iv) loading the mesh onto the device when in position;

(v) deploy and position/anchor the mesh to the MSSL;

(vi) deploy and position/anchor to the MSSL a variety of meshes [Prosima, Prolift (Gynecare), Elevate (AMS), Pinnacle (Boston Scientific)].

The device head was angled at 30 degrees to the tuberosity bone. Mesh loading onto the device was difficult and the device was too close to the thigh when positioned at the above angle. It was deemed that a curved device will accommodate mesh/suture/anchor loading without the above described limitations.

Results

The results are presented in Table 1 below.

TABLE 1

| Objective | Results | comments |
|---|---|---|
| Ability to dissect the tissue | In all cadavers dissection of | 10 dissections-5 right and 5 |
| with the present device | tissue was successful | left. |
| (non-curved). | | |
| Ability to dissect on 2 sides: | Dissection was successful | |
| right and left, starting at the | | |
| high posterior virginal wall | | |
| and passing through the | | |
| posterior pelvic floor | | |
| compartment; 2 dissections | | |
| in each cadaver. | | |
| Ability to load the mesh | Loading the mesh was | Loading is difficult and too |
| onto the present device | successful | close to the vagina which |
| when in-situ. | | makes it hard to maneuver |
| Ability to deploy and place | 3 different meshes were used | |
| or fix different meshes such | Prolift and Prosima | |
| as: Prosima, Prolift | (Gynecare) | |
| (Gynecare), Elevate (AMS), | IVS tunneler | |
| Pinnacle (Boston Scientific) | (Tyco/Covidian) | |
| to the MSSL. | | |
| Ability to place a suture into | A suture was placed into the | Pull out force should be |
| the MSSL. | MSSL successfully with the | studied |
| | present device | |

### Conclusions

Table 2 below summarizes the operability of the present device and the solutions proposed to some of the problems encountered during the procedures.

TABLE 2

| Observations | proposed solution |
|---|---|
| Introducing the device at the right plane and angle | modify the device body and introduce curvature opposite to |
| is a bit problematic due to space limitation (i.e. leg) | the pelvic curve and articulating head, such that the head |
| | articulates opposite to the device curvature |
| The articulation operation in some cases cause the | modify the device body and introduce curvature opposite to |
| device to move away from the pelvic path | the pelvic curve and articulating head, such that the head |
| | articulates opposite to the device curvature |
| | Two articulation steps the first to Ischial spine 15 degrees and |
| | second to MSSL 30 degrees |
| Loading the suture/mesh into the device was | Extend the device by 10 cm |
| difficult and too close to the vagina | |
| The articulation head is not stiff enough and can | Change the pivot link mechanism to stiffen articulation |
| bend when pushing against the pelvic tissue | |
| Notches are difficult to see during the procedure | Move the notches to be on the working cannel side or around |
| | the entire device. |
| The device can rotate during the procedure and | Fix the device to have only two directions right and left with |
| change the direction of articulation | no option for rotation |
| Successful identification of Para rectal space | Continue as is |
| The device reach the pelvic side wall in all cases | Continue as is |
| The device reached the Ischial spine in all cases | Continue as is |
| The device reached the MSSL in 90% of the cases | See 1 and 2 above |
| The average distance to the Pelvic side wall is 7.3 cm | The current instruction of 7.5 is ok |
| The average distance to the Ischial spine is 8.95 cm | Change the distance at our landmark to 1.5 cm instead of 2 |
| The average distance to MSSL is 10.56 cm | Change the distance at our landmark to 1.5 cm instead of 2 |
| Laparotomy measurements showed the mesh/suture | Attachment of meshes/suture worked well |
| is located at safe distances away from crucial | |
| organs. | |
| Articulation trigger is not easy to operate | Move the trigger to a different location an industrial designer |
| ergonomically | will get involved |
| Dissection handle location is not ideal | industrial designer will get involved |
| ergonomically | |
| Couldn't insert the push rod for mesh placement | Enable the rod to be inserted while articulated |
| while the articulating head is articulated | |
| Jaws cover to prevent tissue entrapment is not | Move away from silicone and add a special third fix jaw in |
| optimal | the middle |
| Jaws are too long and the opening is too wide - this | Shortening the jaws and reducing the opening angle |
| makes dissection a bit more difficult | |

The study clearly demonstrated that the present device is highly useful in pelvic floor procedures and that further improvements to the present device such as a curved body positioned opposite to the articulating head, a trigger for actuating pivoting of the head which is located at the front of the device and a dissection head that includes a finger-like extension flanked by jaws which move sideways, forward and then back (movement illustrated in FIGS. 17a-c) can further improve the efficacy of the present device in pelvic floor procedures.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In the claims:

1. A surgical device comprising:
(a) an elongated device body having a proximal end and a distal end, said elongated device body being curved along at least a portion of its length; and
(b) a tissue dissecting head being pivotally attached to said distal end and having at least one pair of movable jaws configured for blunt tissue dissection and for preventing tissue trapping within said tissue dissecting head.

2. The surgical device of claim 1, wherein a radius of curvature of said at least a portion of said elongated device body is 9-11 cm along a sector of 45 degrees.

3. The surgical device of claim 1, wherein said at least one pair of movable jaws includes a finger-like extension positionable between said movable jaws when in an open position.

4. The surgical device of claim 1, further comprising an actuator attached to said proximal end of said elongated device body, said actuator being for opening and closing said at least one pair of movable jaws and for pivoting said tissue dissection head.

5. The surgical device of claim 4, wherein said actuator includes a manual trigger for separately triggering said opening and closing of said at least one pair of movable jaws and for said pivoting of said tissue dissection head.

6. The surgical device of claim 5, wherein said manual trigger is actuatable to a first position for opening and closing of said at least one pair of movable jaws and to a second position for pivoting of said tissue dissection head.

7. The surgical device of claim 6, wherein said first and said second positions lie along a single actuation path.

8. The surgical device of claim 1, wherein said elongated device body includes a longitudinal channel extending to said distal end of said elongated device body, wherein an opening of said channel at said distal end is exposed when said tissue dissection head is pivoted away from said elongated device body and/or when said at least one pair of jaws is an open position.

9. The surgical device of claim 8, wherein said longitudinal channel includes a longitudinal slot through said elongated device body.

10. A surgical device comprising:
(a) an elongated device body having a proximal end and a distal end and being curved along at least a portion of its length
(b) a tissue dissecting head being attached to said distal end and having at least one pair of movable jaws configured for blunt tissue dissection; and
(c) a longitudinal channel extending to said distal end of said elongated device body.

11. The device of claim 10, wherein said longitudinal channel is slotted along a length thereof.

12. The surgical device of claim 10, further comprising an element for preventing trapping of tissue between said at least one pair of movable jaws.

13. The surgical device of claim 11, wherein said element is a finger-like extension positionable between said movable jaws when in an open position.

14. The surgical device of claim 10, wherein said tissue dissecting head is pivotally attached to said distal end.

15. The surgical device of claim 14, further comprising an actuator attached to said proximal end of said elongated device body, said actuator being for opening and closing said at least one pair of movable jaws and for pivoting said tissue dissection head.

16. The surgical device of claim 15, wherein said actuator includes a manual trigger for separately triggering said opening and closing of said at least one pair of movable jaws and for said pivoting of said tissue dissection head.

17. The surgical device of claim 16, wherein said manual trigger is actuatable to a first position for opening and closing of said at least one pair of movable jaws and to a second position for pivoting of said tissue dissection head.

18. The surgical device of claim 17, wherein said first and said second positions lie along a single actuation path.

19. The surgical device of claim 14, wherein an opening of said channel at said distal end is exposed when said tissue dissection head is pivoted away from said elongated device body.

20. A system for pelvic floor repair comprising the surgical device of claim 1 or 10 and a suture attached to a tissue anchor.

21. The system of claim 20, further comprising a mesh attached to said suture.

22. A method of providing tissue access to an anatomical landmark comprising:
(a) providing a surgical device including:
(i) an elongated device body having a proximal end and a distal end and being curved along at least a portion of its length; and
(ii) a tissue dissecting head being attached to said distal end and having at least one pair of movable jaws configured for blunt tissue dissection;
(b) actuating said at least one pair of movable jaws of said surgical device to blunt dissect tissue in a first path; and
(c) actuating said at least one pair of movable jaws of said surgical device while optionally pivoting said dissection head with respect to said elongated device body to blunt dissect tissue in a second path thereby providing tissue access to the anatomical landmark.

23. The method of claim 22, wherein the anatomical landmark is a sacrospineous ligament.

24. The method of claim 23, wherein said first path is from a posterior vaginal wall.

25. The method of claim 24, wherein said second path and said first path are in the same dissection plane.

26. The method of claim 24, wherein said first path is from said posterior vaginal wall in a direction of an ischial spine.

27. The method of claim 26, wherein said second path is from an ischial spine region in a direction of said sacrospineous ligament.

28. A method of repairing a pelvic floor disorder comprising:
(a) providing a surgical device including:
(i) an elongated device body having a proximal end and a distal end and being curved along at least a portion of its length;
(ii) a tissue dissecting head being attached to said distal end and having at least one pair of movable jaws configured for blunt tissue dissection; and
(iii) a longitudinal channel extending to said distal end of said elongated device body;
(b) actuating said at least one pair of movable jaws of said surgical device to blunt dissect tissue in a first path;
(c) actuating said at least one pair of movable jaws of said surgical device while optionally pivoting said dissection head with respect to said elongated device body to blunt dissect tissue in a second path thereby providing tissue access to the anatomical landmark;
(d) exposing a distal opening of said longitudinal channel;
(e) using said longitudinal channel to deliver a tissue repair device to the anatomical landmark thereby repairing the pelvic floor disorder.

29. The method of claim 28, wherein the pelvic floor disorder is central pelvic apical prolapse.
